(19) United States
(12) Patent Application Publication
(10) Pub. No.: US 2025/0161118 A1
(43) Pub. Date: May 22, 2025

(54) WOUND DRESSING

(71) Applicant: CARL FREUDENBERG KG, Weinheim (DE)

(72) Inventors: Marc PEHR, Laudenbach (DE); Bernd SCHLESSELMANN, Weinheim (DE); Samuel DUNCKER-RAKOW, Bensheim (DE)

(21) Appl. No.: 18/866,607

(22) PCT Filed: Mar. 28, 2023
(86) PCT No.: PCT/EP2023/057955

§ 371 Date: Nov. 18, 2024

(30) Foreign Application Priority Data
May 19, 2022 (DE) 10 2022 112 586.6

(51) Int. Cl. A61F 13/01; A61L 15/28; A61L 15/42; A61L 15/60; D04H 1/407; D04H 1/4282; D04H 1/435; D04H 1/4382; D04H 1/4391; D04H 1/482

(52) U.S. Cl. CPC A61F 13/01017; A61F 13/01012; A61F 13/01038; A61F 13/01042; A61L 15/28; A61L 15/42; A61L 15/60; D04H 1/407; D04H 1/4282; D04H 1/435; D04H 1/43825; D04H 1/43835; D04H 1/43918; D04H 1/482; D10B 2321/08; D10B 2401/022; D10B 2401/06; D10B 2509/022

(57) ABSTRACT

A wound dressing including a needle-punched nonwoven. The needle-punched nonwoven of the wound dressing is 30% to 80% by weight, based on a total weight of the needle-punched nonwoven, superabsorbent fibers. The needle-punched nonwoven of the wound dressing is 70% to 20% by weight, based on the total weight of the needle-punched nonwoven, self-crimped fibers.

### CROSS REFERENCE TO RELATED APPLICATIONS

This application is a U.S. National Phase application under 35 U.S.C. § 371 of International Application No. PCT/EP2023/057955, filed on Mar. 28, 2023, and claims benefit to German Patent Application No. DE 10 2022 112 586.6, filed on May 19, 2022. The International Application was published in German on Nov. 23, 2023, as WO 2023/222291 A1 under PCT Article 21(2).

### FIELD

The invention relates to a wound dressing and a method for its manufacture. The invention further relates to the use of a needle-punched nonwoven containing superabsorbent fibers for the manufacture of a wound dressing.

### BACKGROUND

Highly absorbent nonwovens containing superabsorbent fibers are generally known. They are often used as components in wound dressings as they have a high absorption capacity and retention capacity relative to wound fluid. In addition, they are adaptable due to their textile properties and can therefore contribute to a high wear comfort of the wound dressing.

However, the disadvantage of such nonwovens containing superabsorbent fibers is that they are not reversibly expandable, i.e. they do not, or only barely, react elastically to tension. This means that forces caused by patient movement, for example, can lead to a loss of adhesion or slippage of the wound dressing. This applies in particular if the wound dressing is used on or in the vicinity of a joint. If the components of the wound dressing have different elasticities, this may lead also to a delamination of the components.

A further disadvantage of nonwovens containing superabsorbent fibers is that they shrink from contact with humidity during storage and when liquid is absorbed during application. If such a nonwoven is used as a component in a wound dressing that also contains other, non-shrinking layers, the shrinkage of the nonwoven may lead to wrinkling or to deformation of the entire wound dressing.

In addition, superabsorbent fibers, especially if they are made of polyacrylate or polymethacrylate, generally lose their integrity during absorption of aqueous liquids, and the hydrogel formed in the process is not incorporated into the nonwoven stably against mechanical influences. Even minor mechanical stress can cause gel particles to detach from the nonwoven.

Various approaches have already been proposed to provide the absorbent components of a wound dressing with elastic properties. However, these often require the use of elastomeric materials or complex manufacturing processes. Furthermore, some of these approaches only provide elasticity in one dimension. Thus for example, WO2010/035017A1 describes a thread reinforcement with elastic yarn. However, the resulting elasticity is only effective in the longitudinal direction. EP3666295A1 describes the bonding of an absorbent nonwoven to a fabric of elastomeric material. This is effort-intensive and the elasticity achieved in this way is not homogeneous across the thickness of the resulting material.

EP3285705B1 describes a wound dressing with a fiber layer which is provided with groups of incisions and thus exhibits increased flexibility. However, the layer made deformable in this way shows only slight elastic recovery.

Also known are elastic nonwovens made from latently self-crimping fibers. Thus for example, U.S. Pat. No. 5,454,142A describes a method for manufacturing a nonwoven from fibers with a mechanical crimp and a latent crimp (self-crimp). Thereby the nonwoven is heated to form the latent crimp. The nonwovens produced in this way exhibit very good elastomeric and foam-like compressibility and elasticity properties. The fibers are made of thermoplastic materials and especially of polyester, and have little or no absorbent properties. They are therefore not suitable for absorbent components in wound dressings.

### SUMMARY

In an embodiment, the present disclosure provides a wound dressing comprising a needle-punched nonwoven. The needle-punched nonwoven of the wound dressing is 30% to 80% by weight, based on a total weight of the needle-punched nonwoven, superabsorbent fibers. The needle-punched nonwoven of the wound dressing is 70% to 20% by weight, based on the total weight of the needle-punched nonwoven, self-crimped fibers.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 illustrates a stress-strain diagram characterizing elasticity of a wound dressing test specimen; and

FIG. 2 illustrates comparative examples of embodiments of the present disclosure before and after testing.

### DETAILED DESCRIPTION

In an embodiment, the present invention provides a wound dressing which combines a high elasticity with a good absorption capacity, which is at the same time stable against mechanical influences after absorption of liquid, which exhibits no or only slight shrinkage from contact with liquid or with air humidity during storage and which exhibits gel-blocking properties. A method for manufacturing the wound dressing as well as the use of a non-woven for its manufacture are provided.

In an embodiment, a wound dressing is provided, the wound dressing comprising a needle-punched nonwoven which contains:
30% to 80% by weight, preferably 40% to 75% by weight, even more preferable 45% to 70% by weight, in particular 55% to 65% by weight, based on the total weight of the needle-punched nonwoven, superabsorbent fibers;
70% to 20% by weight, preferably 60% to 25% by weight, even more preferably 55% to 30% by weight, in particular 45% to 35% by weight based on the total weight of the needle-punched nonwoven, self-crimped fibers.

The wound dressing according to the present disclosure is characterized by a high elasticity combined with a good absorption capacity. At the same time, it is stable against mechanical influences and, it exhibits no or only slight shrinkage and gel-blocking properties after absorption of liquid or contact with air humidity during storage.

Without specifying a mechanism, it is assumed that these advantageous properties are due to the fact that the self-crimped fibers are able to incorporate the superabsorbent fibers particularly well into their framework structure and thus stabilize them. As a result, the gel formed by the superabsorbent fibers during the absorption of aqueous liquids can be stably bound into the needle-punched nonwoven against mechanical influences. It was particularly surprising thereby that a good incorporation of the gel in combination with elastic recovery can already be achieved with a minority proportion of self-crimped fibers. Furthermore, it was found that the wound dressing according to the present disclosure exhibits gel-blocking properties. This is advantageous in wound dressings because it hinders the transport of fluid in a vertical direction beyond the wound edge and thus exposing the uninjured skin to less fluid, which in turn prevents the occurrence of maceration. It is assumed that this effect is caused by the fact that the self-crimping fibers lead to a compaction of the needle-punched nonwoven, whereby less free volume is available for fluid transport.

Self-crimped fibers are fibers that are manufactured from self-crimping fibers (also known as latent crimping fibers). Their crimp can be obtained by thermally triggering the latent crimping ability of the self-crimping fibers used as starting material. Preferably, the needle-punched nonwoven is therefore thermally treated. An advantage of the use of self-crimping fibers is that they give the needle-punched nonwoven a high degree of elasticity.

Preferably, the self-crimped fibers have fiber materials selected from the group of polyamides, for example polyamide 6, polyamide 6,6 or polyamide 11, polyesters, for example polyethylene terephthalate (PET) or polybutylene terephthalate (PBT), polybutylene succinate (PBS), and polyolefins, for example polypropylene (PP) or polyethylene (PE), or mixtures or copolymers thereof.

Preferred self-crimped fibers are made of self-crimping fibers which have at least two components, wherein one component has a different shrinkage behavior under thermal treatment than the other component and wherein the arrangement of the components induces a three-dimensional deformation of the fiber, for example in the form of a spiral or helix, or in an irregular structure deviating from straight.

Likewise preferred self-crimped fibers are manufactured from self-crimping fibers in which there is a different shrinkage behavior under thermal treatment of at least one fiber component compared to at least one other fiber component. Advantageously, this is achieved by the use of different polymers that differ in their morphology. For example, a homopolymer can be used as one component and a copolymer as another component. Alternatively, a difference in morphology can be created by different molecular weights, stereochemistry, degree of branching or cross-linking. Typically, the self-crimping fibers have a first fiber component and a second fiber component.

Preferably, the fiber components of the self-crimping fibers, in particular the first component and the second fiber component, comprise fiber materials which are selected independently from each other from the group of polyamides, for example polyamide 6, polyamide 6,6 or polyamide 11, polyesters, for example polyethylene terephthalate (PET) or polybutylene terephthalate (PBT), polybutylene succinate (PBS), and the polyolefins, for example polypropylene (PP) or polyethylene (PE), or mixtures or copolymers thereof.

The self-crimped fibers and/or the self-crimping fibers used as starting material can be present as bicomponent fibers in which the fiber components are present, for example, in side-by-side arrangement with an overall round cross-section, or in eccentric core-sheath arrangement with an overall round cross-section. However, a crimping potential can also be achieved with other cross-sections and bicomponent shapes known in the prior art.

As presented above, the self-crimped fibers used according to the present disclosure are made from self-crimping fibers whose crimp can be obtained by thermally releasing their latent crimping ability. This has the advantage that it is possible to carry out the crimping only after the manufacture of the nonwoven, which in turn is advantageous because it enables the formation of elastic recovery and at the same time a good incorporation of the superabsorbent fibers takes place.

Irrespective of their self-crimping, the self-crimped fibers can have a mechanical crimp. The providing of the fibers with the mechanical crimping preferably is carried out during the manufacture of the fibers, usually using stuffing boxes or saw-tooth gears. This mechanical crimping serves to improve the processability of the latently crimped fibers during the nonwoven manufacturing.

According to the present disclosure, both the self-crimped fibers and the superabsorbent fibers are preferably staple fibers. This is advantageous because needle-punched nonwovens can be manufactured with a high volume, which contributes to provide the needle-punched nonwoven with absorbent properties. Thereby the staple fiber length of the self-crimped fibers and/or the superabsorbent fibers is, independently of one another other, preferably between 32 and 80 mm, preferably between 38 and 70 mm, and particularly preferably from 40 to 60 mm. The titer of the self-crimped fibers and/or the superabsorbent fibers is, independently of one another, preferably between 1.7 and 20 dtex, particularly preferably between 2.0 and 10 dtex.

Superabsorbent fibers are fibers formed from a material having the property of absorbing aqueous liquids under formation of a hydrogel. Preferred superabsorbent fibers have a free absorption capacity of aqueous NaCl solution (0.9% by weight), measured as defined in the Measurement Methods section, of at least 20 g/g, for example from 20 g/g to 70 g/g, preferably from at least 30 g/g to 60 g/g, particularly preferably from 40 g/g to 50 g/g.

Preferred superabsorbent fibers contain polymers selected from the group consisting of polyacrylic acid and/or polymethacrylic acid, graft copolymers of unsaturated carboxylic acids, modified cellulose, from gel-forming polysaccharides other than modified cellulose, and mixtures thereof.

Preferred polyacrylic acids and/or polymethacrylic acids are selected from acrylic acid polymers, methacrylic acid polymers, acrylic acid/methacrylic acid copolymers and mixtures thereof. Thereby the acid groups of the polymers are preferably partially neutralized to sodium salts. It is also preferred that there is a crosslinking between the polymer chains. Superabsorbent fibers that can be used according to the present disclosure are available, for example, under the trade name OASIS SAF, manufactured by TECHNICAL ABSORBENTS LTD.

In another preferred embodiment, the superabsorbent fibers, in particular the polyacrylate-containing superabsorbent fibers, have a two-layer structure which comprises an outer superabsorbent layer which has been provided with carboxyl groups by means of a hydrolyzing treatment and an inner layer of polyacrylonitrile. Such fibers are described in U.S. Pat. No. 4,366,206A and are available under the trade name LANSEAL, manufactured by JAPAN EXLAN CO., LTD.

Likewise suitable superabsorbent fibers have a compound of the copolymer of unsaturated carboxylic acids and of disaccharides, oligosaccharides and/or polysaccharides. Such fibers are described, for example, in U.S. Pat. No. 4,788,237A.Co.

Preferred graft copolymers of unsaturated carboxylic acids are copolymers of unsaturated carboxylic acids and of disaccharides, oligosaccharides and/or polysaccharides.

Also suitable superabsorbent fibers are manufactured by chemical modification of cellulosic fibers. Preferred cellulosic fibers are carboxymethylated cellulosic fibers, in particular cellulosic fibers as described in EP0680344. Also preferred cellulosic fibers are carboxyethylated cellulosic fibers, in particular fibers as described in WO2011022740A1. Also preferred cellulosic fibers are fibers obtained by alkylsulfonation of cellulosic fibers, in particular fibers as described in EP2196224A1.

Preferred modified cellulose is selected from carboxymethylated cellulose, carboxyethylated cellulose, alkylsulfonated cellulose and mixtures thereof.

Also suitable superabsorbent fibers contain polymers selected from gel-forming polysaccharides, in particular from alginate, pectin, chitosan or hyaluronic acid and mixtures thereof. Particularly suitable superabsorbent fibers of this type are described in EP1085912A1.

In addition to the superabsorbent fibers and the self-crimped fibers, the needle-punched nonwoven can contain other fibers, preferably selected from fibers containing one or more of the following fiber raw materials: polyolefins, cellulose, regenerated cellulose, in particular viscose, polyamides, polyacrylonitriles, elastanes, polyvinyl chlorides, polylactides, polyglycolides, polyesteramides, polycaprolactones, polyhexamethylene terephthalates, polyhydroxybutyrates, polyhydroxyvalerates, animal and/or vegetable natural fibers and/or polyesters.

Preferred further fibers are hydrophilic fibers. According to the present disclosure, the term “hydrophilic fibers” is understood to mean that the fiber in question has a fiber material on the surface which has a surface energy, measured according to DIN 55660-2:2011-12, of >35 mN/m. Particularly preferred hydrophilic fibers are selected from hydrophilic fibers which contain one or more fiber raw materials selected from cellulose, preferably regenerated cellulose, in particular viscose.

An advantage of hydrophilic fibers is that their use leads to particularly good wettability of the needle-punched nonwoven, even after thermal treatment. This in turn is advantageous for vertical liquid transport.

If present, the other fibers are preferably present in a proportion of 5 wt. % to 30 wt. %, even more preferably 10 wt. % to 20 wt. %, based on the total weight of the needle-punched nonwoven.

According to the present disclosure, the wound dressing comprises a needle-punched nonwoven. Nonwovens are understood to be textile fabrics which have been formed from fibers, filaments or cut yarns into a fibrous web and bonded using a process other than weaving, warp knitting or knitting.

The distinction between nonwovens and other textile fabrics is defined in the standard DIN EN ISO 9092:2019-08. The forming of fibers into a fibrous web can be carried out using various technical processes, such as carding, the airlaid process, the wetlaid process or melt spinning. Preferred are carded fibrous webs. Fibrous webs can also be pre-bonded for better processing.

In a preferred embodiment of the invention, the superabsorbent fibers and the self-crimped fibers are distributed, preferably homogeneously, in the needle-punched nonwoven. This means that the fibers are not arranged in the form of different layers in the nonwoven. As is known to the skilled person, such a homogeneous distribution can be achieved, for example, by carding the fibers during the manufacture of the fibrous web. The homogeneous distribution has the advantage that the superabsorbent fibers can be incorporated particularly well into their framework structure by the self-crimped fibers.

The nonwoven according to the present disclosure is a needle-punched nonwoven. Needle-punched nonwovens are fabrics that are formed by the bonding of a fibrous web by needle-punching. The needles used for this purpose have points which create a vertical puncture channel in the fibrous web, as well as notches which grip fibers or fiber bundles and draw them into the vertical puncture channel during the puncturing movement. As a result, needle-punched nonwovens have regions of high fiber density and partially vertical orientation of the fibers, as well as regions of lower fiber density between the puncture channels. Needle-punched nonwovens have the advantage that the needle-punching creates zones in which the needle-punched nonwoven is fixed and the fiber between the fixed zones is drawn together by crimping. This leads to a high elastic deformability. Preferably, the needle-punched nonwoven has a needle density of 50 to 150 punctures/cm², preferably 70 to 120 punctures/cm², in particular 90 to 110 punctures/cm².

As presented above, the needle-punched nonwoven is preferably thermally treated. Thereby the latent crimping ability of the self-crimping fibers used as the starting material can be triggered. As a rule, the needle-punched nonwoven is thermally treated at temperatures of 160° C. to 200° C., preferably 180° C. to 190° C.

In a further preferred embodiment of the invention, the wound dressing has at least one further fiber layer which is arranged on at least one side of the needle-punched nonwoven. Preferably, the further fiber layer is connected to the needle-punched nonwoven by needle-punching. This is advantageous because the connection by needle-punching forms puncture channels that enable accelerated liquid transport from the further fiber layer into the needle-punched nonwoven. Preferably, the additional fiber layer is formed as a nonwoven.

Particularly preferably, the at least one further fiber layer is connected to the needle-punched nonwoven in that at least one further fibrous web has been applied to the fibrous web used as starting material for manufacturing the needle-punched nonwoven and the fibrous webs have been needle-punched together.

If present, the at least one further fiber layer is hydrophilic in one embodiment. According to the present disclosure, the term “hydrophilic” means that the fiber layer has a surface energy measured according to DIN 55660-2:2011-12, of >35 mN/m.

This is advantageous because such a fiber layer absorbs liquid particularly quickly and transfers it to the inside of the nonwoven.

In a further embodiment, the at least one further fiber layer is hydrophobic. According to the present disclosure, the term “hydrophobic fiber layer” is understood to mean that the fiber layer has a surface energy measured according to DIN 55660-2:2011-12, of <35 mN/m. This is advantageous because such a fiber layer in direct contact with a wound does not lead to a sticking of the wound dressing to the wound.

In a further preferred embodiment, the at least one additional fiber layer is thermoplastic. This is advantageous because such a fiber layer can be smoothed, allowing loose fiber ends to be incorporated. This is additionally advantageous because such a fiber layer enables thermal lamination with further conventional components of a wound dressing.

In a further preferred embodiment, the at least one additional fiber layer has a thermoplastic elastomeric configuration. This is advantageous because the use of such a fiber layer has a beneficial effect on the thermoplastic and elastic properties of the wound dressing.

In a further preferred embodiment, the at least one additional fiber layer has fibers with hydrophilic properties in combination with fibers that exhibit thermoplastic behavior at the temperature at which the crimping of the self-crimped fibers was triggered. This is advantageous because the fiber layer formed in this way can transfer aqueous liquids and distribute them over an area before they are absorbed by the layer containing superabsorbent fibers. This favors the rapid absorption of large quantities of wound exudate.

In a further preferred embodiment of the invention, the wound dressing, in particular the needle-punched nonwoven, is finished with at least one antimicrobial active ingredient, for example selected from the group consisting of silver, silver salts such as silver nitrate or silver sulfate, iodine-containing compounds, biguanidines, for example chlorhexidine or polyhexamethylene biguanidine (“PHMB”), quaternary ammonium compounds, for example benzalkonium chloride, or octenidine hydrochloride and mixtures thereof. If present, the wound dressing, in particular the needle-punched nonwoven, has the antimicrobial active ingredient preferably in an amount of 0.1 g to 10 g per square meter, preferably 0.5 g to 5 g per square meter.

Furthermore, the wound dressing, in particular the needle-punched nonwoven, can comprise various additives. Preferred additives are selected from pharmacological active ingredients or medications, in particular analgesics, anti-inflammatory agents, wound-healing agents, hemostatic agents, enzymes, amino acids, antioxidants, peptides, peptide sequences, polysaccharides, in particular chitosan, growth factors, in particular purines, pyrimidines, odor-adsorbing additives, in particular activated carbon and/or processing aids, in particular surface-active substances, wetting agents, avivages, antistatic agents and/or mixtures thereof.

If present, the wound dressing, in particular the needle-punched nonwoven, has the other additives in an amount of 0.1 g to 10 g per square meter, preferably 0.5 g to 5 g per square meter.

Furthermore, the wound dressing can contain further conventional components such as hydrophilic foams, for example polyurethane foams, liquid-impermeable backings and/or adhesive masses.

The wound dressing and/or the needle-punched nonwoven according to the present disclosure is characterized by low shrinkage from contact with liquid or air humidity during storage. According to the present disclosure, the wound dressing and/or the needle-punched nonwoven preferably has a shrinkage, measured as defined in the section on measuring methods, of 0% to 10% after 24 hours, even more preferably of 0% to 5%.

The wound dressing and/or the needle-punched nonwoven according to the present disclosure is further characterized by a high absorption. Preferably according to the present disclosure, the wound dressing and/or the needle-punched nonwoven has an absorption capacity, measured as defined in the Measurement Methods section, for 0.9% aqueous sodium chloride solution of 1000% to 5000%, or from 1000% to 2500%.

The wound dressing and/or the needle-punched nonwoven according to the present disclosure is characterized by a high elasticity. Preferably, the plastic portion of the deformation ε(pl) of the wound dressing and/or of the needle-punched nonwoven, determined as defined in the Measuring Methods section, is longitudinally and/or transversely, particularly preferably, longitudinally and transversely, less than 12%, preferably less than 10%, and, particularly preferably, less than 8%.

The wound dressing according to the present disclosure and/or the needle-punched nonwoven is further characterized by good gel-blocking properties, which is reflected in a low capillary height of NaCl solution. Preferably, the capillary height of 0.9 wt. % NaCl solution of the wound dressing according to the present disclosure and/or the needle-punched nonwoven, determined as defined in the Measuring Methods section, is longitudinally and/or transversely, preferably longitudinally and transversely, less than 30 mm, preferably less than 20 mm.

Further preferably, the wound dressing according to the present disclosure and/or the needle-punched nonwoven is sterile. This means that the wound dressing and/or the needle-punched nonwoven has been sterilized, preferably using a standard sterilization method. Expediently, the wound dressing has been sterilized by at least one of the following methods: high-energy radiation, in particular γ-radiation, by heat, under pressure, by steam, in particular in an autoclave, or by reactive chemicals, such as ethylene oxide.

It is further preferred that the wound dressing according to the present disclosure and/or the needle-punched nonwoven is calendered.

The wound dressing according to the present disclosure can advantageously be manufactured by a method comprising the following steps:
1) Manufacturing and/or providing of a fibrous web comprising 30% by weight to 80% by weight, preferably 40% to 75% by weight, more preferably 45% to 70% by weight, in particular 55% to 65% by weight, based on the total weight of the fibrous web, of superabsorbent fibers, and 70% to 20% by weight, preferably 60% to 25% by weight, even more preferably 55% to 30% by weight, in particular 45% to 35% by weight, based on the total weight of the fibrous web of self-crimping fibers;
2) Bonding of the fibrous web obtained in step 1) to a needle-punched nonwoven by needle-punching;
3) Triggering the self-crimping of the self-crimping fibers of the bonded fibrous web obtained in step 2), preferably by thermal treatment, whereby a wound dressing is obtained.

The process steps are preferably carried out one after the other.

Preferred embodiments of the method according to the present disclosure comprise the preferred embodiments described in relation to the wound dressing according to the present disclosure.

Preferably, the manufacture of the fibrous web is carried out in step 1) in such a way that a homogeneous distribution of the fiber types takes place. This can be achieved by carding, for example. Accordingly, preferably a carded fibrous web is provided.

In another preferred embodiment, the fibrous web is laid down with a cross-lapper.

If further fibers are used, they are preferably mixed homogeneously with the superabsorbent fibers and the self-crimping fibers during the manufacture of the fibrous web.

Self-crimping fibers are used as a starting material for the manufacture of the fibrous web. Irrespective of their self-crimping ability, the self-crimping fibers can have a mechanical crimp. The formation of the mechanical crimp is preferably carried out during the manufacture of the fibers, usually using stuffing boxes or saw-tooth gears. This mechanical crimping is advantageous because it leads to better processability of the self-crimping fibers.

In step 2), the fibrous web obtained in step 1) is bonded to a needle-punched nonwoven by needle-punching. The needle-punching is preferably carried out with 50 to 150 punctures/cm², preferably with 70 to 120 punctures/cm², in particular with 90 to 110 punctures/cm².

In step 3), the self-crimping of the self-crimping fibers of the bonded fibrous web obtained in step 2) is triggered, preferably by thermal treatment. As a rule, the heating is carried out at temperatures of 160° C. to 200° C., preferably 180° C. to 190° C.

Preferably, the self-crimping of the self-crimping fiber is triggered by treating the needle-punched nonwoven in a continuous dryer equipped with an oven belt, having overfeed with sag in the machine direction. This is advantageous because the needle-punched nonwoven manufactured in this way exhibits uniform elasticity in the longitudinal and transverse direction.

In a further preferred embodiment, the self-crimping of the self-crimping fiber is triggered by treating the needle-punched nonwoven in a continuous dryer with a stenter frame. Preferably, the width of the tensioned material is reduced by bringing the stenter frame together during the treatment. This is advantageous because the needle-punched nonwoven produced in this way exhibits selective elasticity in the transverse direction. This results in good processability on kitting machines for wound dressings.

In a further preferred embodiment, the self-crimping of the self-crimping fibers is triggered by treating the needle-punched nonwoven in a continuous dryer with a stenter frame, whereby the width of the tensioned material is reduced by bringing the stenter frame together during the treatment, and at the same time the distance between the fastening points of the needle-punched nonwoven on the stenter frame is reduced by bringing the individual fastening elements of the stenter frame together in the machine running direction.

This is advantageous because the needle-punched nonwoven manufactured in this way exhibits different degrees of elasticity in the longitudinal and transverse direction.

In addition, a kitting and/or post-treatment of the individual components of the procedure, in particular the wound dressing and/or the needle-punched nonwoven, can be carried out.

A preferred post-treatment comprises the application of a wetting agent. Expediently, the wetting agent is applied to the wound dressing and/or the needle-punched nonwoven.

A further preferred post-treatment comprises sterilization. Expediently, the wound dressing and/or the needle-punched nonwoven is sterilized, in particular by at least one of the following methods: high-energy radiation, in particular γ-radiation, by heat, under pressure, by steam, in particular in an autoclave, or by reactive chemicals, such as ethylene oxide.

Another preferred post-treatment comprises calendering. Preferably, the wound dressing and/or the needle-punched nonwoven is calendered. The calendering is preferably carried out using at least one calender roller with a textured surface. This is advantageous because an additional bonding can be obtained and/or a structured surface can be created.

Another provided post-treatment is a post-bonding, a coating and/or a chemical finishing, preferably of the wound dressing and/or the needle-punched nonwoven.

Another provided post-treatment is the addition of processing aids and/or additives. If a thermal treatment is carried out in step 3), this post-treatment is preferably carried out after the thermal treatment. Processing aids that can be used include, for example, fabric softener, antistatic agents, surfactants, stabilizers and/or lubricants.

In an embodiment, the present invention further provides the use of a needle-punched nonwoven comprising:
30% to 80% by weight, preferably 40% to 75% by weight, even more preferably 45% to 70% by weight, in particular 55% to 65% by weight, based on the total weight of the needle-punched nonwoven, superabsorbent fibers;
70% to 20% by weight, preferably 60% to 25% by weight, even more preferably 55% to 30% by weight, in particular 45% to 35% by weight based on the total weight of the needle-punched nonwoven, self-crimped fibers, for the production of a wound dressing.

Preferred embodiments of the use according to the present disclosure comprise the preferred embodiments described with respect to the wound dressing according to the present disclosure, mutatis mutandis.

Embodiments of the invention are explained in more detail below with reference to several non-limiting examples.

### Embodiment Example 1

Three fibrous webs with a basis weight of 150 g/m² with different blending ratios of superabsorbent fiber (Oasis 112) and latent self-crimping fiber (examples 1 to 3) were produced on a carding machine with cross-lapper, as well as another fibrous web made only of latent self-crimping polyester fiber (Comparative Example 1). The fibrous webs were bonded by needle-punching with a needle loom equipped with needles from the manufacturer Groz-Beckert, type R222 G, with a puncturing depth of 10 mm and 100 punctures/cm². The needle-punched nonwovens obtained in this way were passed through a continuous dryer at 185° C. with a 5% overfeed so that the material could shrink freely by triggering latent self-crimping in the transverse direction.

The reference sample with superabsorbent fiber was a needle-punched nonwoven made of 60% by weight superabsorbent fiber (Oasis 112) and 40% by weight of a mechanically crimped polyester fiber that does not have self-crimping properties (Comparative Example 2).

| | Superabsorbent Fiber | Self-crimping Fiber |
|---|---|---|
| Example 1 | 20% by weight | 80% by weight |
| Example 2 | 40% by weight | 60% by weight |
| Example 3 | 60% by weight | 40% by weight |
| Comparative Example 1 | 0% by weight | 100% by weight |
| Comparative Example 2 | 60% by weight | 0% by weight |

The elasticity and absorption capacity of 0.9% sodium chloride solution was determined for the needle-punched nonwovens produced in this way.

| | Absorption | ε(pl) t.s. |
|---|---|---|
| Example 1 | 570% | 6.0% |
| Example 2 | 1120% | 7.0% |
| Example 3 | 1890% | 7.7% |
| Comparative Example 1 | 460% | 4.9% |
| Comparative Example 2 | 2100% | 15.9% |

It could be shown that all examples according to the present disclosure exhibit a high elastic recovery in combination with a high absorption of NaCl solution.

The shrinkage of the needle-punched nonwovens produced in this way, Example 3 and Comparative Example 2, from contact with air humidity was determined as described in the Measurement Methods section.

| | 2 h | 4 h | 6 h | 24 h |
|---|---|---|---|---|
| Example 3 | 0% | 1.8% | 2.0% | 3.3% |
| Comparative Example 2 | 2.0% | 6.0% | 8.7% | 11.3% |

It could be shown that Example 3 exhibits significantly less shrinkage than Example 2, which contains the same proportion of superabsorbent fibers (Oasis 112).

### Embodiment Example 2

Another needle-punched nonwoven was produced using the same method as Example 3, with 60% by weight of the superabsorbent fiber Lanseal FK.

| | Superabsorbent | Self-crimping |
|---|---|---|
| | Fiber | Fiber |
| Example 4 | 60% Lanseal | 40% |

In Example 3, Example 4 and Comparative Example 2, the capillary height of NaCl solution was determined in the longitudinal and transverse direction.

| | Capillary Height in mm | Capillary Height in mm |
|---|---|---|
| | (lengthwise) | (transverse) |
| Example 3 | 19 | 22 |
| Example 4 | 14 | 15 |
| Comparative | 34 | 34 |
| Example 2 | | |

It could be shown that both Example 3 and Example 4 have a significantly lower capillary height than Example 2, which contains the same proportion of superabsorbent fibers (Oasis 112).

### Embodiment Example 3

The incorporation of the gel formed by the superabsorbent fibers of Example 3 and Comparative Example 2 was compared. For this purpose, a 10×10 cm test piece was punched out, placed in deionized water for 5 min, subsequently placed for 5 min on a grid with a weight having a base area of 10×10 cm and a weight of 4100 kg, and then placed on an adhesive tape (CMC 10966 Polyester Tape from the manufacturer CMC Klebetechnik) and pressed on twice with a roller with a weight of 1580 kg. Finally, the test pieces were removed from the adhesive tape. The gel that emerged from the gelled needle-punched nonwoven was assessed by hand and the appearance of the test pieces was compared.

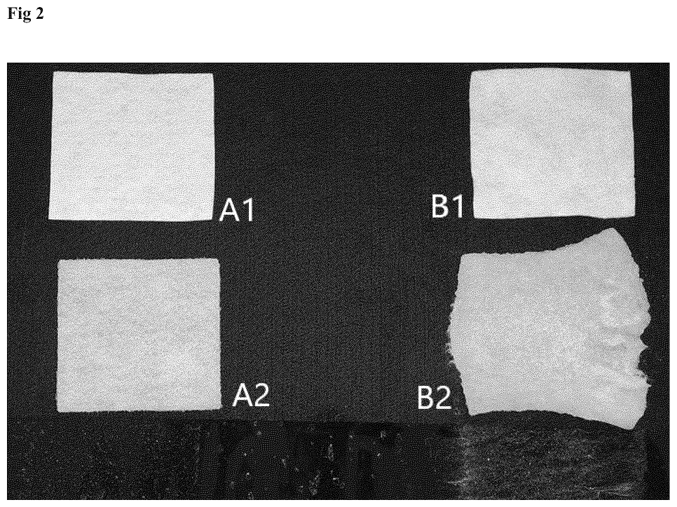

In Example 3, significantly less tangible gel escaped from the surface than in Comparative Example 2. In addition, Example 3 retained its shape much better after removal from the adhesive tape, as shown in FIG. 2 (A1: Example 3 before the test; A2: Example after the test; B1: Comparative Example 2 before the test; B2: Comparative Example 2 after the test).

### Measurement Methods

### Free Absorption Capacity of Fibers:

1 g of the superabsorbent fiber sample is weighed (W₁) placed into a tea bag (acrylic/polyester gauze with fine mesh) and the bag is immersed for one hour in an excess amount of NaCl solution 0.9% by weight (tempered to 20° C.). The excess solution is then removed by hanging the bag until no more liquid drips off. The tea bag is weighed (W₂). The procedure is repeated with an empty tea bag and weighed (W0) and the swelling capacity is calculated according to the following equation:

$$Free absorption capacity = ( W 2 - W 1 - W 0 ) / W 1$$

Shrinkage on Contact with Air Humidity:

The shrinkage is determined by punching out 10.0 cm×10.0 cm (area 1) pieces from the wound dressing and/or the nonwoven and storing them in a climate chamber at a temperature of 37° C. and a relative air humidity of 45%. The punched out and stored pieces are removed from the climate chamber after a specified time has passed and the size of the pieces is measured (area 2). The shrinkage of the punched-out pieces can then be calculated using the following formula:

$$shrinkage [ \% ] = 100 - \frac{area 2 [ {cm}^{2} ]}{area 1 [ {cm}^{2} ]} * 100$$

### Absorption Capacity:

The absorption capacity is determined by punching out and weighing (weight 1) 10.0 cm×10.0 cm pieces from the wound dressing and/or non-woven and immersing them in a 0.9% aqueous sodium chloride solution (tempered to 20° C.). The punched out and soaked pieces are removed from the solution and drained for 2 min. They are then weighed again (weight 2). The absorption capacity is then calculated using the following formula:

$$absorption capacity [ \% ] = \frac{weight 2 [ g ] - weight 1 [ g ]}{weight 1 [ g ]} * 100$$

### Elasticity:

To characterize the elasticity, a rectangular 50 mm wide test specimen is punched out of the wound dressing and/or the non-woven and tensioned with a tensioning length of 200 mm in a tension testing machine (e.g. type Z020/SN3A.02SO1 from the manufacturer Zwick/Roell). The test specimen is pulled at a speed of 10 mm/min until a pre-load ε(vor) of 0.2 N is measured. The length when ε(vor) is reached is regarded as the initial length L(0). From this point onwards, it is pulled further at a constant speed of 100 mm/min to a distance of L(0)*120%, and subsequently brought back again at 100 mm/min until the measured force has dropped to 0.2 N again. The associated distance relative to L(0) in the stress-strain diagram is interpreted as the plastic portion of the deformation ε(pl). A wound dressing and/or a non-woven with high elasticity is characterized by a low value of ε(pl). All measurements are carried out 5 times and the results are averaged.

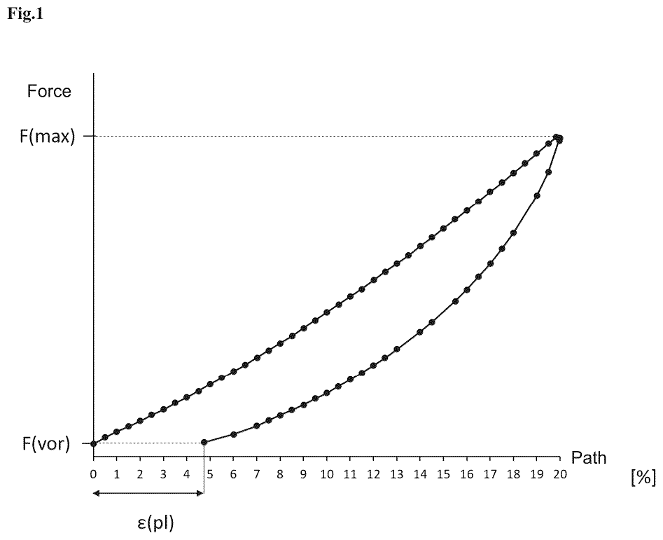

An exemplary stress-strain diagram for illustration is shown in FIG. 1.

### Capillary Height Nacl Solution

The NaCl 0.9% by weight capillary height is determined in accordance with DIN EN ISO 9073-06 using an apparatus consisting of a liquid tub, vertical scales with a scale graduation of 1 mm intervals and clamps whose height can be adjusted relative to the liquid tub. The plastic tub is filled to the level of the overflow with 0.9% by weight NaCl solution up to the level of the overflow and the zero point of the scales is adjusted to the filling height of the NaCl solution.

A test specimen measuring 250 mm×30 mm is punched out. One end of the test specimen is provided with two holes and weighted down with a metal rod which is passed through the holes. The test specimen is attached to the clamp so that the end weighted with the metal rod points downwards and hangs above the surface of the liquid.

At the beginning of the measurement, the setting clamp is lowered so far that the end weighted with the metal rod is completely lowered into the liquid. After 5 minutes, the height of the liquid front is read off using the scales. If the liquid front is uneven, the highest point of the capillary height is read off.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A wound dressing comprising a needle-punched nonwoven that is:
30% to 80% by weight, based on a total weight of the needle-punched nonwoven, superabsorbent fibers, and
70% to 20% by weight, based on the total weight of the needle-punched nonwoven, self-crimped fibers.

2: The wound dressing according to claim 1, wherein the needle-punched nonwoven is thermally treated.

3: The wound dressing according to claim 1, wherein the self-crimped fibers comprise fiber materials which are selected from the group of polyamides, polyesters, polyolefins, and mixtures or copolymers thereof.

4: The wound dressing according to claim 1, wherein the self-crimped fibers are produced from self-crimping fibers which have at least two components,
wherein one component of the at least two components has a different shrinkage behavior under thermal treatment than the other component of the at least two components, and
wherein an arrangement of the at least two components is configured to induce a three-dimensional deformation of the self-crimped fibers under thermal treatment and/or wherein the self-crimped fibers are manufactured from self-crimping fibers in which there is a different shrinkage behavior under thermal treatment of at least one fiber component with respect to at least one other fiber component.

5: The wound dressing according to claim 1, wherein the self-crimped fibers and/or the self-crimping fibers used as starting material are present as bicomponent fibers.

6: The wound dressing according to claim 1, wherein the self-crimped fibers and the superabsorbent fibers are staple fibers,
wherein a staple fiber length of the self-crimped fibers and/or the superabsorbent fibers independently of each other is between 32 and 80 mm and/or the titer of the self-crimped fibers and/or the superabsorbent fibers independently of each other is between 1.7 and 20 dtex.

7: The wound dressing according to claim 1, wherein the superabsorbent fibers contain polymers selected from the group consisting of polymers of unsaturated carboxylic acids, graft copolymers of unsaturated carboxylic acids, modified cellulose, gel-forming polysaccharides other than modified cellulose, and mixtures thereof.

8: The wound dressing according to claim 1, wherein the needle-punched nonwoven also comprise further fibers selected from fibers including one or more of the following fiber raw materials: polyolefins, cellulose, regenerated cellulose, polyamides, polyacrylonitriles, elastanes, polyvinyl chlorides, polylactides, polyglycolides, polyesteramides, polycaprolactones, polyhexamethylene terephthalates, polyhydroxybutyrates, polyhydroxyvalerates, animal and/or vegetable natural fibers and/or polyesters.

9: The wound dressing according to claim 8, wherein the further fibers are hydrophilic fibers including one or more fiber raw materials selected from cellulose and regenerated cellulose.

10: The wound dressing according to claim 8, wherein the further fibers are present in a proportion of 5% to 30% by weight based on the total weight of the needle-punched nonwoven.

11: The wound dressing according to claim 1, wherein the needle-punched nonwoven has a needle density of 50 to 150 punctures/cm².

12: The wound dressing according to claim 1, wherein the wound dressing has at least one further fiber layer which is arranged on at least one side of the needle-punched nonwoven, and wherein the at least one further fiber layer is connected to the needle-punched nonwoven by needle-punching.

13: The wound dressing according to claim 12, wherein the at least one further fiber layer is hydrophilic.

14: The wound dressing according to claim 1, wherein the wound dressing and/or the needle-punched nonwoven has a shrinkage from contact with air humidity after 24 hours of 0% to 10% and/or the needle-punched nonwoven has an absorption capacity for 0.9% aqueous sodium chloride solution from 1000% to 5000% and/or a plastic portion of the deformation ε(pl) of the wound dressing and/or the needle-punched nonwoven is longitudinally and/or transversely less than 12%, and/or the capillary height of 0.9% by weight NaCl solution of the wound dressing and/or the needle-punched nonwoven is less than 30 mm, longitudinally and/or transversely, preferably longitudinally and transversely.

15: A method of manufacturing a wound dressing, the method comprising:
1) manufacturing and/or providing a fibrous web containing 30% to 80% by weight, based on a total weight of the fibrous web, superabsorbent fibers and 70% to 20% by weight, based on the total weight of the fibrous web, self-crimping fibers;
2) bonding the fibrous web obtained in step 1) into a needle-punched nonwoven by needle-punching;
3) triggering self-crimping of the self-crimping fibers of the bonded fibrous web obtained in step 2), preferably by thermal treatment, wherein wound dressing is obtained.

16. (canceled)
